(19) United States
(12) Patent Application Publication
(10) Pub. No.: US 2021/0038569 A1
(43) Pub. Date: Feb. 11, 2021

(54) COMPOSITIONS AND METHODS FOR TREATING PIGMENTATION DISORDERS

(71) Applicant: Timber Pharmaceuticals, Inc., Woodcliff Lake, NJ (US)

(72) Inventor: Zachary Rome, Nyack, NY (US)

(73) Assignee: Timber Pharmaceuticals, Inc., Woodcliff Lake, NJ (US)

(21) Appl. No.: 16/978,183

(22) PCT Filed: Mar. 4, 2019
(86) PCT No.: PCT/US19/20557

§ 371 Date: Sep. 3, 2020

Related U.S. Application Data
(60) Provisional application No. 62/639,767, filed on Mar. 7, 2018.

(51) Int. Cl. A61K 31/4155; A61Q 17/04; A61P 17/00; A61K 8/29; A61K 8/28; A61K 8/27; A61K 8/36; A61K 8/46

(52) U.S. Cl. CPC A61K 31/4155; A61Q 17/04; A61P 17/00; A61K 8/466; A61K 8/28; A61K 8/27; A61K 8/36; A61K 8/29

(57) ABSTRACT

The present invention relates to local or topical compositions containing a therapeutically effective amount of a selective endothelin-A (ET-A) receptor antagonist or inhibitor, preferably sitaxentan (also known as sitaxsentan), and pharmaceutically acceptable salts thereof. The compositions are useful for treating a patient that has a pigmentation disorder or irregularity.

### FIELD OF THE INVENTION

The present invention relates to local or topical compositions containing a therapeutically effective amount of a selective endothelin-A (ET-A) receptor antagonist or inhibitor, preferably sitaxentan (also known as sitaxsentan), and pharmaceutically acceptable salts thereof. The compositions are useful for treating a patient that has a pigmentation disorder or irregularity.

### BACKGROUND OF THE INVENTION

In humans, normal skin can range from a “pale white” that always burns and does not tan to a “dark brown or black” that never burns and always tans darkly. Although compounds like carotenoids and hemoglobin may contribute to skin tone, melanin is the primary pigment responsible for the various colorations found in animal skin, hair, and eyes. In normal physiology, the amount and type of melanin produced is primarily dependent upon genetic factors.

Melanin is produced in specialized skin cells called melanocytes, located at the junction of the epidermis and dermis. Once synthesized, melanin is transferred from the melanocytes to the keratinocytes, the predominant cell type in the epidermis. In a cycle lasting approximately 40-60 days, the keratinocytes migrate suprabasally from the basal layer of the epidermis to eventual become corneocytes in the stratum corneum. One's resulting skin color is, therefore, determined by the amount and type of melanin that is produced along with any factors that may impact its transport through the epidermis.

Disorders or irregularities of skin pigmentation are common. They can be broadly categorized as conditions of hypopigmentation (lightening of the skin) or hyperpigmentation (darkening of the skin). Disorders of hyperpigmentation usually result from an increase in melanin production or from an increase in the density of active melanocytes. Some, but not all, examples of disorders of hyperpigmentation are postinflammatory hyperpigmentation (PH), erythema dyschromicum perstans (EDP), lichen planus pigmentosus (LPP), and melasma. Some, but not all, examples of other disorders or irregularities of pigmentation are lentigo, age spots, freckling, vitiligo, albinism, acanthosis nigricans, incontinentia pigmenti, progressive pigmentary purpura, xeroderma pigmentosum, café au lai spots or macules, cholasma, liver spots, Addison Disease, melanocytic naevi, sebhorreic keratosis, melanoma, basal cell carcinoma, pityriasis alba, pityriasis versicolor, idiopathic guttate hypomelanosis, progressive macular hypomelanosis, and urticarial pigmentosa. Additionally, pigmentation changes can be caused by external factors including, but not limited to, drug reactions, infections, burns, and chemical damage.

The irregular distribution of skin pigmentation is often aesthetically undesirable for a person. In some instances, the target area of treatment is the area that is itself irregular in pigmentation. For example, if one has a spot of darkened skin, they might try to apply a treatment to that spot to lighten it. In other instances, the target area of treatment is the area surrounding the irregularity in pigmentation. For example, if one has a spot of lightened skin, they might try to apply a treatment to the surrounding areas to lighten the surrounding area and “even out” the irregularity. In other instances, there might not be a particular area of irregular pigmentation and one might simply want lighter or darker skin for cosmetic reasons. For example, it has been shown that there is a strong consumer demand for treatments that can lighten skin coloration and “even out” tone, especially in the Asian population and markets.

There are some products that are used to try and lighten or “bleach” the darkened areas; however, there are many concerns with their safe and effective use. For example, one of the most common compounds used for skin lightening is hydroquinone. Hydroquinone alone is considered by the Food and Drug Administration (FDA) to be an “unapproved drug”, i.e. it has not been found by FDA to be safe and effective, and the labeling accompanying such products has not been approved by FDA. Additionally, in 2006, the FDA determined that hydroquinone could not be Generally Recognized as Safe and Effective (GRASE) and that it cannot be ruled out as a potential carcinogen. See https://www.fda.gov/OHRMS/DOCKETS/98fr/78n-0065-npr0003.pdf and https://www.fda.gov/AboutFDA/CentersOffices/OfficeofMedicalProductsandTobacco/CDER/ucm203112.htm. The only instance in which hydroquinone has been approved by FDA is when it is used in combination with fluocinolone acetonide (a corticosteroid) and tretinoin (a retinoid). In this instance, it is only indicated for the short-term treatment of moderate to severe melasma of the face. It is not indicated for the maintenance treatment of melasma or for other areas of the body and side effects can be common and significant. For example, in the clinical trials for the product, some, but not all, adverse effects included: erythema in 41% of patients, desquamation in 38% of patients, burning in 18% of patients, dryness in 14% of patients, pruritus in 11% of patients, and acne in 5% of patients. See https://dailymed.nlm.nih.gov/dailymed/drugInfo.cfm?setid=a35fa709-5eb5-4429-b38f-f1e0019bf0ee.

It is apparent from the foregoing that there is an ongoing need for developing safe and effective treatments for disorders, irregularities, and/or cosmetic preferences of skin pigmentation. Further, it is apparent that there are significant concerns with the use of current skin lightening treatments and that other mechanisms of achieving skin lightening should be considered. It has been surprisingly found in the present invention that selective modulation of the endothelin axis may provide a new mechanism for treating disorders or irregularities of pigmentation, especially hyperpigmentation.

The endothelins (ET-1, ET-2, and ET-3) constitute a family of 21 amino acid peptides that act on two distinct high-affinity receptor subtypes, endothelin-A (ET-A) and endothelin-B (ET-B). Of these three peptides, ET-1 has been the most studied and is believed to be the most representative peptide of the axis. It can be induced in endothelial cells by many factors including mechanical stimulation, various hormones, and pro-inflammatory cytokines. ET-1 stimulates cardiac contraction and the growth of cardiac myocytes, regulates the release of vasoactive substances (it is a potent vasoconstrictor), stimulates smooth muscle mitogenesis, and may control inflammatory responses by promoting the adhesion and migration of neutrophils and by stimulating the production of pro-inflammatory cytokines. It has also been implicated in cancer progression, regulating the proliferation and migration of tumor cells and acting as a pro-angiogenic factor and inducer of stromal reaction. See https://www.ncbi.nlm.nih.gov/pubmed/27266371. Given their broad activity, therapeutically controlling the endothelins has been an area of interest for potential treatments for many different pathological conditions. However, the outcomes of these investigations have largely been disappointing, and therapeutically controlling the endothelin axis has proven to be a complicated, nuanced, and challenging task. For example, within five years of the discovery of the endothelin axis, orally available endothelin receptor antagonists became available and their effects were evaluated in clinical trials for cardiovascular diseases, heart failure, pulmonary arterial hypertension, resistant arterial hypertension, stroke, subarachnoid hemorrhage, kidney diseases, and various cancers. Aside from treating pulmonary arterial hypertension, the results of most clinical trials for other indications were either neutral or negative, leading to the discontinuation of endothelin-receptor antagonist programs in many pharmaceutical companies. See https://www.ncbi.nlm.nih.gov/pubmed/27266371. One success, though, was bosentan, a dual (i.e. a non-selective) ET-NET-B receptor antagonist, that was developed for and is now FDA approved (in a tablet form) to treat pulmonary arterial hypertension (PAH).

Current research suggests that there is either no benefit to selective antagonism of ET-A vs. ET-B or that ET-B selective antagonism is preferable. For example, in PAH, both ET-A-selective and dual ET-A/ET-B antagonists have been approved by FDA, and yet a close analysis of their clinical outcomes revealed that it was not possible to identify a clinically relevant advantage for one class of drug over the other. It was, however, observed that patients on ET-A-selective drugs experienced more significant adverse events, particularly fluid retention. This observation was not unique. Among other examples, Phase 3 clinical trials for two different ET-A-selective antagonists (one for diabetic neuropathy and the other for cancer) led to early study termination due to water retention or increased mortality. See https://www.ncbi.nlm.nih.gov/pubmed/27266371. Research efforts were looking for selective endothelin inhibitors, particularly compounds selective for inhibiting ET-A versus ET-B. Sitaxentan, a selective ET-A antagonist, was developed as an oral tablet for treating pulmonary arterial hypertension (PAH). Sitaxanten gained regulatory approval in Europe, but was voluntarily withdrawn from the market within five years based on emerging safety concerns, particularly those associated with liver toxicity. Consequently, sitaxsentan never gained FDA approval in the United States.

As a further example, in vivo and in vitro investigations exploring the effect of microvascularization on skin pigmentation concluded that endothelial cells promote pigmentation through ET-B receptor activation. Regazzetti et al. explain that in melanocytes the stimulation of ET-B activates protein kinase C, which, in turn, stimulates extracellular signal-regulated kinase (ERK)½ and p38, mitogen-activated protein kinases that are implicated in the phosphorylation of microphthalmia-associated transcription factor and the upregulation of tyrosinase, respectively. See http://www.jidonline.org/article/S0022-202X(15)60177-4/fulltext. Tyrosinase is the rate-limiting enzyme for controlling the production of melanin. These findings, therefore, suggest that melanogenesis and the promotion of pigmentation can be counteracted through antagonizing ET-B, and not ET-A. In view of these prior art teachings, it was therefore surprising that we discovered that sitaxentan was superior to bosentan in reducing the melanin content in both Caucasians and African-American melanocytes. It was also surprising to discover that sitaxsentan was significantly less cytotoxic to human skin cells than bosentan. Additionally, we have discovered a means of treating pigmentation disorders or irregularities with local or topical, non-systemic dosages of sitaxentan. For example, when sitaxentan was used in pulmonary arterial hypertension, oral dosing often reached 100 mg/day, resulting in unwanted systemic side effects. In contrast, the local or topical compositions of the present invention can provide a benefit with plasma levels that are significantly less than those obtained from oral dosing of an ET-A inhibitor. Further, the novel approach of treating pigmentation disorders or irregularities through the local or topical application of endothelin antagonists provides a means of avoiding the well-known and significant systemic side effects that have prevented the previous utility of these compounds.

It is apparent from the foregoing that there is an ongoing need for developing safe and effective treatments for pigmentation disorders or irregularities. Therapeutically controlling the endothelins may offer important treatment opportunities but attempts thus far have been unsuccessful. It has been surprisingly found in the present invention that the local or topical application of a selective ET-A receptor antagonist may be safely administered to treat pigmentation disorders or irregularities. It has surprisingly been found in the present invention that the selective endothelin-A receptor inhibitor, sitaxentan, can be safely and effectively administered locally or topically to treat pigmentation disorders or irregularities.

### SUMMARY OF THE INVENTION

The present invention relates to methods of use and compositions for the local or topical application of selective ET-A receptor antagonists or inhibitors for the treatment of pigmentation disorders or irregularities.

The present invention is based on the surprising discovery that sitaxentan, a highly selective ET-A receptor antagonist, and not bosentan, a non-selective ET-A/ET-B receptor antagonist, was significantly more effective than a vehicle control at reducing melanin content in human melanocytes.

### BRIEF DESCRIPTION OF THE DRAWINGS

FIG. 1 shows experimental results for male normal human dermal fibroblasts (NHDFs) which were stimulated with 50 ng/mL transforming growth factor-β1 (TGF-β1) for 48 hours. Viability was measured comparing sitaxentan (SIT, 100 μM), against bosentan (BOS, 100 μM as a comparator compound) and vehicle control (VC), and reported as relative fluorescence units (RFUs) on the y-axis. Statistical significance is indicated as follows: *p<0.05 to control, #p<0.05 between experimental groups, n=6. One-Way ANOVA using Tukey's honest significant difference (HSD) post-hoc analysis.

FIG. 2 shows experimental results for male normal human dermal fibroblasts (NHDFs) which were stimulated with 50 ng/mL transforming growth factor-β1 (TGF-β1) for 48 hours. Cytotoxicity was measured comparing sitaxentan (SIT, 100 μM), against bosentan (BOS, 100 μM as a comparator compound) and vehicle control (VC), and reported as relative fluorescence units (RFUs) on the y-axis. Statistical significance is indicated as follows: #p<0.05 between experimental groups, n=6. One-Way ANOVA using Tukey's honest significant difference (HSD) post-hoc analysis.

FIG. 3 shows experimental results for male normal human dermal fibroblasts (NHDFs) which were stimulated with 50 ng/mL transforming growth factor-β1 (TGF-β1) for 48 hours. Apoptosis was measured comparing sitaxentan (SIT, 100 μM), against bosentan (BOS, 100 μM as a comparator compound) and vehicle control (VC), and reported as relative light units (RLUs) on the y-axis. Statistical significance is indicated as follows: *p<0.05 to control, #p<0.05 between experimental groups, n=6. One-Way ANOVA using Tukey's honest significant difference (HSD) post-hoc analysis.

FIGS. 4A, 4B, 4C, and 4D show experimental results for primary normal human adult melanocytes (Caucasian and African American) treated 24 hours with 30 μM and 100 μM of sitaxentan (SIT), bosentan (BOS) drug, and vehicle control (VC). Results are reported as melanin content as determined by UV spectroscopy in absorbance (y-axis units) at 400 nm. FIG. 4A shows results for the 30 μM treatments on Caucasian melanocytes. FIG. 4B shows results for the 30 μM treatments on African American melanocytes. FIG. 4C shows results for the 100 μM treatments on Caucasian melanocytes. FIG. 4D shows results for the 100 μM treatments on African American melanocytes. Statistical significance is indicated as follows: *p<0.05 to control, n=3. One-Way ANOVA using Tukey's honest significant difference (HSD) post-hoc analysis.

### DETAILED DESCRIPTION OF THE INVENTION

The present invention relates to a method for treating hyperpigmentation or a pigmentation disorder or irregularity comprising locally or topically applying a therapeutically effective amount of a selective endothelin-A (ET-A) receptor antagonist or inhibitor to a mammal in need thereof

In another aspect, the present invention relates to a method wherein the selective endothelin-A (ET-A) receptor antagonist or inhibitor has a selectivity of at least two-fold over endothelin-B (ET-B).

In another aspect, the present invention relates to a method wherein the selective endothelin-A (ET-A) receptor antagonist or inhibitor has a selectivity of at least five-fold over endothelin-B (ET-B).

In another aspect, the present invention relates to a method wherein the selective endothelin-A (ET-A) receptor antagonist or inhibitor has a selectivity of at least ten-fold over endothelin-B (ET-B).

In another aspect, the present invention relates to a method wherein the selective endothelin-A (ET-A) receptor antagonist or inhibitor has a selectivity of at least 100-fold over endothelin-B (ET-B).

In another aspect, the present invention relates to a method wherein the selective endothelin-A (ET-A) receptor antagonist or inhibitor has a selectivity of at least 1000-fold over endothelin-B (ET-B).

In another aspect, the present invention relates to a method wherein the selective endothelin-A (ET-A) receptor antagonist or inhibitor has a selectivity of at least 5000-fold over endothelin-B (ET-B).

In another aspect, the present invention relates to a method wherein the selective endothelin-A (ET-A) receptor antagonist or inhibitor is sitaxentan or a pharmaceutically acceptable salt thereof.

In another aspect, the present invention relates to a method wherein the mammal is a human patient.

In another aspect, the present invention relates to a method wherein the pigmentation disorder is selected from postinflammatory hyperpigmentation, erythema dyschromicum perstans, lichen planus pigmentosus, melasma, lentigo, age spots, freckling, vitiligo, albinism, acanthosis nigricans, incontinentia pigmenti, progressive pigmentary purpura, Xeroderma pigmentosum, café au lai spots or macules, cholasma, liver spots, Addison Disease, melanocytic naevi, sebhorreic keratosis, melanoma, basal cell carcinoma, pityriasis alba, pityriasis versicolor, idiopathic guttate hypomelanosis, progressive macular hypomelanosis, urticarial pigmentosa, and pigmentation changes caused by drug reactions, infections, burns, and chemical damage, and combinations thereof.

In another aspect, the present invention relates to a method wherein the pharmaceutically acceptable salt is selected from an alkali metal salt, an alkaline earth metal salt, and an ammonium salt.

In another aspect, the present invention relates to a method wherein the alkali metal salt is selected from lithium, sodium, and potassium.

In another aspect, the present invention relates to a method wherein the alkali metal salt is sodium.

In another aspect, the present invention relates to a method wherein the pharmaceutically acceptable salt is sitaxentan sodium.

In another aspect, the present invention relates to a method wherein the selective endothelin-A (ET-A) receptor antagonist or inhibitor is applied at least one daily.

In another aspect, the present invention relates to a method wherein the selective endothelin-A (ET-A) receptor antagonist or inhibitor is applied at least twice daily.

In another aspect, the present invention relates to a method wherein the selective endothelin-A (ET-A) receptor antagonist or inhibitor is applied at least once weekly.

In another aspect, the present invention relates to a method wherein the selective endothelin-A (ET-A) receptor antagonist or inhibitor is applied at least twice weekly.

In another aspect, the present invention relates to a method wherein the selective endothelin-A (ET-A) receptor antagonist or inhibitor is applied at least once daily until the pigmentation disorder or irregularity is treated.

In another aspect, the present invention relates to a method wherein the selective endothelin-A (ET-A) receptor antagonist or inhibitor is applied from a pharmaceutically acceptable composition.

In another aspect, the present invention relates to a method for treating a pigmentation disorder or irregularity, comprising locally or topically applying a pharmaceutically acceptable composition comprising a therapeutically effective amount of a selective endothelin-A (ET-A) receptor antagonist or inhibitor to a mammal in need thereof.

In another aspect, the present invention relates to the use of a selective endothelin-A (ET-A) receptor antagonist or inhibitor in the manufacture of a medicament for local or topical delivery of a therapeutically effective amount of the selective endothelin-A (ET-A) receptor antagonist or inhibitor for treating a pigmentation disorder or irregularity in a mammal in need thereof.

In another aspect, the present invention relates to a composition for local or topical delivery comprising a therapeutically effective amount of a selective endothelin-A (ET-A) receptor antagonist or inhibitor and a pharmaceutically acceptable carrier.

In another aspect, the present invention relates to a composition wherein the selective endothelin-A (ET-A) receptor antagonist or inhibitor has a selectivity of at least two-fold over endothelin-B (ET-B).

In another aspect, the present invention relates to a composition wherein the selective endothelin-A (ET-A) receptor antagonist or inhibitor is sitaxentan or a pharmaceutically acceptable salt thereof.

In another aspect, the present invention relates to a composition wherein the pharmaceutically acceptable salt is sitaxentan sodium.

In another aspect, the present invention relates to a composition further comprising one or more sunscreen actives.

In another aspect, the present invention relates to a composition wherein the sunscreen active is selected from aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, Zinc oxide, and combinations thereof.

In another aspect, the present invention relates to a composition for administration to a mammal.

In another aspect, the present invention relates to a composition wherein said mammal is a human patient.

In another aspect, the present invention relates to a composition in the form of a unit dosage composition.

In another aspect, the present invention relates to a composition comprising about 0.01 to about 1000 mg of sitaxentan or a pharmaceutically acceptable salt thereof, based on the weight of the sitaxentan active.

In another aspect, the present invention relates to a composition comprising from about 0.001% to about 25% by weight sitaxentan or a pharmaceutically salt thereof, based on the weight of the sitaxentan active.

In another aspect, the present invention relates to a composition comprising from about 0.01% to about 10% by weight sitaxentan or a pharmaceutically salt thereof, based on the weight of the sitaxentan active.

In another aspect, the present invention relates to a composition comprising from about 0.1% to about 5% by weight sitaxentan or a pharmaceutically salt thereof, based on the weight of the sitaxentan active.

In another aspect, the present invention relates to a composition comprising from about 0.2% to about 3% by weight sitaxentan or a pharmaceutically salt thereof, based on the weight of the sitaxentan active.

In another aspect, the present invention relates to a composition demonstrating at least one of the following pharmacokinetic parameters selected from a Cₘₐₓ less than about 13 μg/ml, or a Cₘₐₓ less than about 7 μg/ml, or an AUC less than about 40 μg hr/ml.

In another aspect, the present invention relates to a method for preparing a composition according to the present invention.

These and other aspects of the present invention will become apparent from the disclosure herein.

Definitions

As used herein, the following terms and abbreviations have the indicated meanings unless expressly stated to the contrary.

The term “selective” with respect to ET-A antagonist or inhibitor means an ET-A inhibitor which preferentially inhibits ET-A versus ET-B. The selectively for ET-A versus ET-B should be at least two-fold, preferably at least five-fold, more preferably at least ten-fold, more preferably at least 100-fold, more preferably at least 1000-fold, and most preferably at least 5000-fold. Such selectivity can be important for providing the therapeutic benefits of the present invention. A rationale for this selectively, compared to that for a non-selective inhibitor such as bosentan, is negligible inhibition of the beneficial effects of ET-B stimulation, such as nitric oxide production and clearance of endothelin from circulation.

The term “pharmaceutically acceptable” is used herein with respect to the compositions, in other words the formulations, of the present invention, and also with respect to the salts of sitaxentan, i.e. pharmaceutically acceptable salts. The pharmaceutical compositions of the present invention comprise a therapeutically effective amount of sitaxentan and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17^{th} edition, edited by Alfonso R. Gennaro, Mack Publishing Company, Easton, Pa., 17th edition, 1985. Regarding pharmaceutically acceptable salts, these are described below.

The term “subject” means a human patient or animal in need of treatment or intervention for a pigmentation disorder or irregularity.

The term “therapeutically effective” means an amount of sitaxentan needed to provide a meaningful or demonstrable benefit, as understood by medical practitioners, to a subject, such as a human patient or animal, in need of treatment. Conditions, intended to be treated include, for example, a hyperpigmentation disorder. For example, a meaningful or demonstrable benefit can be assessed or quantified using various clinical parameters. The demonstration of a benefit can also include those provided by models, including but not limited to in vitro models, in vivo models, and animal models. An example of such a model is one that uses human melanocytes from Caucasian or African American donors for evaluating the effect of treatments on melanin level. See, https://www.bioalternatives.com/en/cosmetic-claims/skin-pigmentation/.

The term “topical” as used herein with respect to pharmaceutical compositions means a composition that is applied to the skin or mucosal membrane of a subject, such as a human patient. A topical pharmaceutical composition is intended to have an effect at the site of application, i.e. in the tissue beneath the site of application, and does not result in significant drug concentrations in the blood and other tissues. Topical pharmaceutical compositions are in contrast to “transdermal” or “transmucosal” pharmaceutical compositions, which are absorbed through the skin or mucosal membranes and are intended to have a systemic effect in areas of the body away from the site of application. See, http://corporatepharmacy.ca/health-news/topical-vs-transdermal-meds, (2016).

Furthermore, the U.S. Food & Drug Administration has provided a standard for all routes of administration for drugs, i.e. “Route of Administration”. The following definitions are provided by the FDA for topical, transdermal, and transmucosal routes of drug administration.

| | | | FDA | NCI* |
|---|---|---|---|---|
| NAME | DEFINITION | SHORT NAME | CODE | CONCEPT ID |
| TOPICAL | Administration to a | TOPIC | 011 | C38304 |
| | particular spot on the | | | |
| | outer surface of the body. | | | |
| TRANSDERMAL | Administration through the | T-DERMAL | 358 | C38305 |
| | dermal layer of the skin to | | | |
| | the systemic circulation | | | |
| | by diffusion. | | | |
| TRANSMUCOSAL | Administration across the | T-MUCOS | 122 | C38283 |
| | mucosa. | | | |

*National Cancer Institute See, https://www.fda.gov/Drugs/DevelopmentApprovalProcess/FormsSubmissionRequirements/ElectronicSubmissions/DataStandardsManualmonoqraphs/ucm071667.htm.

The term “local” as used herein with respect to pharmaceutical compositions means a route of administration of a composition in which the pharmacodynamic effect is generally contained around the application location and does not result in significant or rapid concentrations in the blood or other tissues. In addition to topical compositions, as defined above, some, but not all, examples of other local routes of administration can include subcutaneous injection and intradermal injection.

The terms “treat,” “treating” or “treatment,” as used herein, include alleviating, abating or ameliorating the condition, e.g. a pigmentation disorder, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The methods of treatment using sitaxentan or a pharmaceutically acceptable salt thereof or the pharmaceutical compositions of the present invention, in various embodiments also include the use of sitaxentan or a pharmaceutically acceptable salt thereof in the manufacture of a medicament for the desired treatment, such as a pigmentation disorder or irregularity.

“ET-A” is an abbreviation for endothelin-A.

“ET-B” is an abbreviation for endothelin-B.

“TGF-β1” is an abbreviation for transforming growth factor-β1.

“NHDF” is an abbreviation for normal human dermal fibroblasts.

Sitaxentan

The present invention utilizes a therapeutically effective amount of a selective endothelin-A (ET-A) receptor antagonist or inhibitor such as sitaxentan or a pharmaceutically acceptable salt thereof, and also a pharmaceutically acceptable carrier for providing local or topical compositions for treating conditions such as a pigmentation disorder or irregularity.

Sitaxentan, also known as sitaxsentan, corresponds to the CAS Registry Number 184036-34-8 and the IUPAC name N-(4-Chloro-3-methyl-5-isoxazolyl)-2-[(2-methyl-4,5-methylenedioxyphenyl)-acetyl]thiophene-3-sulfonamide, and also the code name TBC-11251. Sitaxentan sodium salt, the form of the drug developed for human use, has the CAS Registry Number 210421-64-0. Sitaxentan was developed as an oral tablet for the treatment of pulmonary arterial hypertension (PAH) and was marketed as Thelin® by Encysive Pharmaceuticals until purchased by Pfizer in February 2008. In 2010, Pfizer voluntarily removed sitaxsentan from the market due to emerging safety concerns. http://press.pfizer.com/press-release/pfizer-stops-clinical-trials-thelin-and-initiates-voluntary-product-withdrawal-interes.

The chemical structure for sitaxentan is shown immediately below.

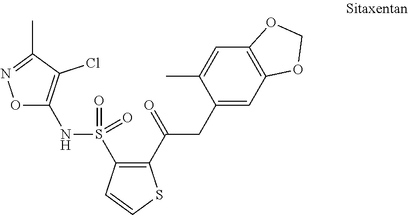 [SMILES: Cc1cc2c(cc1CC(=O)c1sccc1S(=O)(=O)Nc1onc(C)c1Cl)OCO2]

Sitaxentan has the chemical formula C₁₈H₁₅ClN₂O₂S₂ and a molar mass of 454.906 g/mol. The following pharmacokinetic data is reported:

Oral Bioavailability: 70 to 100%

Protein binding: >99%

Metabolism: hepatic (CYP2C9- and CYP3A4-mediated)

Biological half-life: 10 hours

Excretion: renal (50 to 60%), fecal (40 to 50%)

Sitaxentan is described as a small molecule that blocks or inhibits the action of endothelin (ET) on the endothelin-A (ET-A) receptor selectively. This selectivity is reported to be by a factor of 6000 compared to endothelin-B-(ET-B). See, Girgis, R E; Frost, A E; Hill, N S; Horn, E M; Langleben, D; McLaughlin, V V; Oudiz, R J; Robbins, I M; et al. (2007). “Selective endothelin-A receptor antagonism with sitaxsentan for pulmonary arterial hypertension associated with connective tissue disease”. Annals of the rheumatic diseases. 66 (11): 1467-72. doi:10.1136/ard.2007.069609. PMC 2111639 Freely accessible. PMID 17472992. Such selectivity can be important for providing the therapeutic benefits of the present invention.

Pharmaceutically acceptable salts of sitaxentan are useful for the methods and compositions of the present invention. As used herein, “pharmaceutically acceptable salts” refer to derivatives of sitaxentan modified by making salts thereof. Examples of pharmaceutically acceptable salts include, but are not limited to, alkali metal salts, alkaline earth metal salts, and ammonium salts. Examples of alkali metal salts include lithium, sodium, and potassium salts. Examples of alkaline earth metal salts include calcium and magnesium salts. The ammonium salt, NH4⁺. itself can be prepared, as well as various monoalkyl, dialkyl, trialkyl, and tetraalkyl ammonium salts. Also, one or more of the alkyl groups of such ammonium salts can be further substituted with groups such as hydroxy groups, to provide an ammonium salt of an alkanol amine. Ammonium salts derived from diamines such as 1,2-diaminoethane are contemplated herein. The sodium salt of sitaxentan, also called sitaxentan sodium, is useful herein. Lists of suitable salts are found in Remington's Pharmaceutical Sciences, 18th ed. (Mack Publishing Company, 1990).

The pharmaceutically acceptable salts of sitaxentan can be prepared from the parent compound by conventional chemical methods. Generally, such salts can be prepared by reacting the free acid form of the compound with a stoichiometric amount of the appropriate base in water or in an organic solvent, or in a mixture of the two; generally, non-aqueous media like ether, ethyl acetate, ethanol, isopropanol, or acetonitrile are preferred.

Dosages

In one aspect, the present invention comprises a therapeutically effective amount of sitaxentan or a pharmaceutically acceptable salt thereof and a pharmaceutically acceptable carrier.

Compositions, based on a unit dosage can comprise, from about 0.1 mg to about 1000 mg of sitaxentan or a pharmaceutically acceptable salt thereof, based on the weight of the sitaxentan active. Examples of other dosages are 1 mg, 10 mg, 50, mg, 100 mg, and 500 mg of sitaxentan or a pharmaceutically acceptable salt thereof, based on the weight of the sitaxentan active.

Compositions can also be prepared based on weight percentages.

In one embodiment the compositions useful here comprise from about 0.001% to about 25% by weight sitaxentan or a pharmaceutically salt thereof, based on the weight of the sitaxentan active.

In one embodiment the compositions useful here comprise from about 0.01% to about 10% by weight sitaxentan or a pharmaceutically salt thereof, based on the weight of the sitaxentan active.

In one embodiment the compositions useful here comprise from about 0.1% to about 5% by weight sitaxentan or a pharmaceutically salt thereof, based on the weight of the sitaxentan active.

In one embodiment the compositions useful here comprise from about 0.2% to about 3% by weight sitaxentan or a pharmaceutically salt thereof, based on the weight of the sitaxentan active.

For these foregoing compositions comprising a designated amount or weight percentage of the sitaxentan, the amount or weight percentage of the sitaxentan is determined or calculated based on the actual amount of the sitaxentan moiety, which has a molar mass of 454.906, and not including the additional weight provided by any counter ions when a sitaxentan salt is used. In other words, the compositions are based on the amount or weight percentage of the sitaxentan chemical moiety.

Furthermore, because the present invention is related to local or topical compositions and because it is highly desirable to limit systemic exposure, the unit dosage could be formulated to demonstrate at least one of the following pharmacokinetic parameters selected from a Cₘₐₓ less than about 13, μg/ml, or a Cₘₐₓ less than about 7 μg/ml or an AUC less than about 40 μg hr/ml. These pharmacokinetic parameters are based on those reported to the European Medicines Agency for Thelin.

Formulations for Topical Administration

In one embodiment, the compositions or formulations of the present invention comprise a selective ET-A receptor antagonist or pharmaceutically acceptable salt thereof and a pharmaceutically acceptable carrier. These formulations can be made using standard formulation and mixing techniques familiar to one of ordinary skill in the art of pharmaceuticals and formulations.

In one embodiment, the compositions or formulations of the present invention comprise sitaxentan or a pharmaceutically acceptable salt thereof and a pharmaceutically acceptable carrier. These formulations can be made using standard formulation and mixing techniques familiar to one of ordinary skill in the art of pharmaceuticals and formulations.

In one aspect, the pharmaceutical composition is selected from the group consisting of a gel, ointment, lotion, emulsion, cream, foam, mousse, liquid, paste, jelly, tape, spray, suspension, dispersion, or aerosol.

Useful herein are compositions wherein the pharmaceutically acceptable carrier is selected from one or more materials selected from sesame oil, mineral oil, olive oil, petrolatum, water, ethanol, ethanol/water mixtures, isopropanol, isopropanol/water mixtures, dimethyl sulfoxide, and dimethyl isosorbide. Other examples, but not all examples, of pharmaceutical carriers include those selected from oils derived from fruits or vegetables or flowers or nuts or seeds (including, but not limited to, sesame oil, peanut oil, and castor oil), alcohols (including, but not limited to, ethanol, benzyl alcohol, and isopropyl alcohol), dipropylene glycol, ethyl acetate, ethyl lactate, ethyl oleate, glycerin, isopropyl myristate, dimethyl sulfoxide, isopropyl palmitate, medium-chain triglycerides, mineral oil, polyethylene glycol, propylene glycol, tricaprylin, and water. A specific example of a pharmaceutically acceptable carrier is ethanol. These components can be employed and used at levels appropriate for the formulation based on the knowledge of one with ordinary skill in the pharmaceutical and formulation arts. The amounts could range from under 1 percent by weight to up 90 percent or even over 99 percent by weight.

Various additional ingredients can be used in the compositions of the present invention. The compositions can comprise one or more further ingredients selected from a penetration enhancer, a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In one aspect, a penetration enhancer can be included. In another aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

In another aspect, the pharmaceutically acceptable carrier can comprise a material selected from the group consisting of alcohols (including but not limited to ethanol, benzyl alcohol, or isopropyl alcohol), acetone, albumin, oils derived from fruits or vegetables or flowers or nuts or seeds (including but not limited to almond oil, corn oil, cottonseed oil, coconut oil, sesame oil, olive oil, peanut oil, safflower oil, soybean oil, or sunflower oil), benzyl benzoate, butylene glycol, carbon dioxide, castor oil, dibutyl phthalate, diethyl phthalate, diethylene glycol, diethylene glycol monoethyl ether, dimethyl ether, dimethyl phthalate, dimethyl sulfoxide, dimethylacetamide, dipropylene glycol, ethyl acetate, ethyl lactate, ethyl oleate, glycerin, glyceryl monostearate, glycofurol, isopropyl myristate, isopropyl palmitate, light mineral oil, mineral oil, medium-chain triglycerides, methyl lactate, monoethanolamine, octyldodecanol, polyethylene glycol, polyoxyl 35 castor oil, propylene carbonate, propylene glycol, pyrrolidone, triacetin, tricaprylin, triethanolamine, triethyl citrate, triolein, and water, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one penetration enhancer can be selected from the group consisting of alcohols (including but not limited to ethanol, benzyl alcohol, oleyl alcohol, or isopropyl alcohol), diethyl sebacate, diethylene glycol, dimethyl sulfoxide, glyceryl monooleate, glycofurol, isopropyl myristate, isopropyl palmitate, light mineral oil, lauric acid, linoleic acid, menthol, myristic acid, oleic acid, palmitic acid, polyoxyethylene alkyl ethers, polyoxyglycerides, propylene glycol, propylene glycol monolaurate, pyrrolidone, sodium lauryl sulfate, squalane, thymol, tricaprylin, triolein, and transcutol, or a combination thereof. These components can be employed and used at levels appropriate for the formulation based on the knowledge of one with ordinary skill in the pharmaceutical and formulation arts. The amounts could range from under 1 percent by weight to up to 90 percent by weight.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl esthers, polyoxyethylene castor oil derivatives, polyoxyehtylene sorbitan fatty acid esters, polyoxyethylene stearates, polyoxyl 15 hydroxystearate, polyoxylglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, and zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfobutylether β-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

In another aspect, a sunscreen or UV absorbing compound can be included. The sunscreen or UV absorbing compound can be useful to protect the skin from further sun exposure which can cause further pigmentation or UV-induced photo damage. These sunscreen actives are regulated in many countries as over the counter (OTC) drug actives.

In the United States, sunscreen drug products are regulated under the Code of Federal Regulations, Title 21, Volume 5, Chapter I, Subchapter D, Part 352—Sunscreen Drug Products for Over-the-Counter Human Use—revised as of Apr. 1, 2017. The following are the approved OTC sunscreen actives for the United States:

Aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide. Other countries and regions have their own regulatory framework for sunscreen actives. The sunscreen actives and levels that can be formulated into the products where will depend on the target country in which it is to be marketed.

A further list from various geographies of sunscreen actives and maximum amounts which can be formulated by weight are as follows: aminobenzoic acid (PABA) up to 15%, avobenzone up to 3%, cinoxate up to 3%, dioxybenzone up to 3%, homosalate up to 15%, menthyl anthranilate up to 5%, octocrylene up to 10%, octyl methoxycinnamate up to T5%, octyl salicylate up to 5%, oxybenzone up to 6%, padimate O up to 8%, phenylbenzimidazole sulfonic acid up to 4%, sulisobenzone up to 10%, titanium dioxide up to 25%, trolamine salicylate up to 12%, zinc oxide up to 25%, ensulizole up to 4%, homosalate up to 15%, meradimate up to 5%, octinoxate up to 7.5%, octisalate up to 5%, octocrylene up to 10%, oxybenzone up to 6%, padimate O up to 8%.

An overview of sunscreen regulations in the world (as a PDF Download Available), is available from: https://www.researchgate.net/publication/283515177_An_overview_of_sunscreen_regulations_in_the_world [accessed Mar 05 2018].

One of ordinary skill in the pharmaceutical and formulation arts can determine the appropriate levels of the essential and optional components of the compositions of the present invention.

Methods of preparing the sitaxentan compositions are also intended as part of the present invention and would be apparent to one of ordinary skill in the pharmaceutical and formulation arts using standard formulation and mixing techniques.

Methods of Treatment

The present invention utilizes a therapeutically effective amount of sitaxentan or a pharmaceutically acceptable salt thereof and a pharmaceutically acceptable carrier for providing local or topical compositions for treating conditions, disorders, irregularities, and/or cosmetic preferences of skin pigmentation. Such conditions can include postinflammatory hyperpigmentation (PIH), erythema dyschromicum perstans (EDP), lichen planus pigmentosus (LPP), melasma, lentigo, age spots, freckling, vitiligo, albinism, acanthosis nigricans, incontinentia pigmenti, progressive pigmentary purpura, xeroderma pigmentosum, café au lai spots or macules, cholasma, liver spots, Addison Disease, melanocytic naevi, sebhorreic keratosis, melanoma, basal cell carcinoma, pityriasis alba, pityriasis versicolor, idiopathic guttate hypomelanosis, progressive macular hypomelanosis, and urticarial pigmentosa. Additionally, pigmentation changes can be caused by external factors including, but not limited to, drug reactions, infections, burns, and chemical damage, and combinations thereof.

The methods comprise locally or topically applying a therapeutically effective amount of sitaxentan, or a pharmaceutically acceptable salt thereof, to the mammal, such as a human patient, in need thereof. When a human patient is being treated, the composition is applied to the skin of said human.

Various dosing regimens can be prescribed and used based on the skill and knowledge of the physician or other practitioner. In some embodiments, a unit dosage of the composition, as described herein can be applied at least once daily. In other embodiments, a unit dosage of the composition can be applied at least twice daily, or at least once weekly, or at least twice weekly.

Local or topical administration of the composition can be continued in the judgment of the physician or practitioner until the desired therapeutic benefit is achieved, i.e. until the pigmentation disorder or irregularity is treated. In some instances, it can be desirable to continue long term or chronic therapy.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Effect of Sitaxentan on Cell Viability, Cell Cytotoxicity, and Apoptosis in TGF-β1 Induced Human Dermal Fibroblasts

The effect of sitaxentan on cell viability, cell cytotoxicity, and apoptosis was measured in an assay using male normal human dermal fibroblasts induced with TGF-β1 into a profibrotic phenotype. For these assays cells were grown for 48 hours in the presence of vehicle control, sitaxentan, and bosentan. The appropriate assay reagents and measuring techniques were used as indicated herein.

Male normal human dermal fibroblast cells (LLCT FC0024 lot 03869_male fibroblast, 23 year old) were seeded to confluence in 96 well plates in 10% fetal bovine serum (FBS) Dulbecco's modified eagle medium (DMEM).

The cells were washed to remove the FBS, and serum free media was added overnight (O/N).

The cells were then stimulated with 50 ng/mL TGF-β1 and treated.

The following reagents were used for the different assays:

For the cell viability assay:

20 μl of the Viability Reagent (Promega, Cat #G6320) containing both GF-AFC Substrate and bis-AAF-R110 Substrate, for measuring live cell viability, was added and briefly mixed.

For the cell cytotoxicity assay:

20 μl of the Cytotoxicity Reagent (Promega, Cat #G6320) containing both GF-AFC Substrate and bis-AAF-R110 Substrate, for measuring dead cells (cytotoxicity), was added and briefly mixed.

For the apoptosis assay:

100 μl of Caspase-Glo® 3/7 Reagent (Promega, Cat #G6320), for the apoptosis assay, was added and briefly mixed.

The plates for the different assays were then incubated for 30 minutes at 37° C.

Fluorescence was measured at: 400Ex/505Em (Viability) and 485Ex/520Em (Cytotoxicity)

The plate was incubated for 30 minutes at room temperature prior to measurement of luminescence

Data were analyzed using GraphPad Prism 7.

Six to nine replicates were run for each sample.

For cell viability:

The data are presented in Table 1A as the relative fluorescence units (RFUs) as a measure of cell viability.

TABLE 1A
Cell Viability

| | Concentration of Test Material | | | | |
|---|---|---|---|---|---|
| | 1 μM | 3 μM | 10 μM | 30 μM | 100 μM |
| VC | 5528.7 | 5516.3 | 5560.2 | 5400.3 | 5353.7 |
| SIT | 5678.3 | 5639.0 | 5600.5 | 5250.8 | 5171.0 |
| BOS | 5406.8 | 5548.5 | 5521.2 | 5537.0 | 5584.3 |

VC = vehice control,
SIT = sitaxentan,
BOS = bosentan

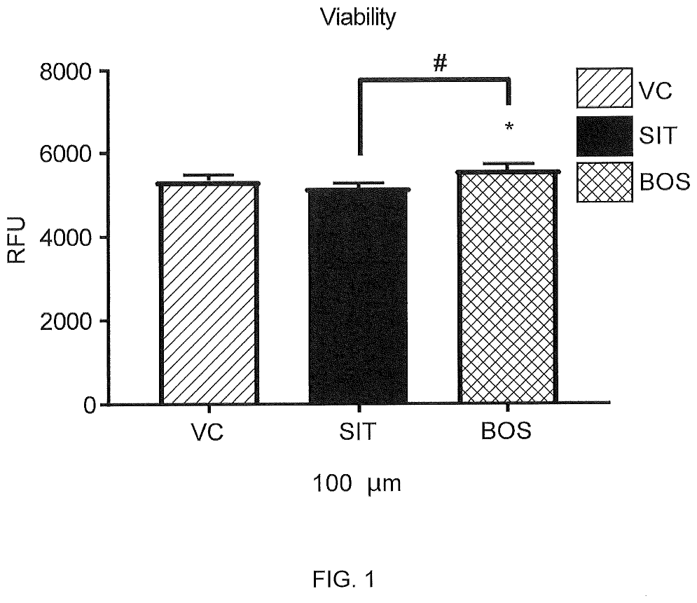

The data from Table 1A for the 100 μM concentrations of vehicle control, sitaxentan, and bosentan are presented as bar graphs with statistical analyses in FIG. 1.

These results show that no significant toxicity was observed for sitaxentan in TGF-β1 induced male normal human dermal fibroblasts at concentrations up to 100 μM.

For cell cytotoxicity:

The data are presented in Table 1B as the relative fluorescence units (RFUs) as a measure of cell cytotoxicity.

TABLE 1B
Cell Cytotoxicity

| | Concentration of Test Material | | | | |
|---|---|---|---|---|---|
| | 1 μM | 3 μM | 10 μM | 30 μM | 100 μM |
| VC | 3837.5 | 3801.2 | 3844.7 | 3496.3 | 3396.8 |
| SIT | 4643.9 | 5058.3 | 4741.7 | 3837.5 | 2911.3 |
| BOS | 4256.9 | 3954.2 | 3764.0 | 4607.5 | 3929.0 |

VC = vehice control,
SIT = sitaxentan,
BOS = bosentan

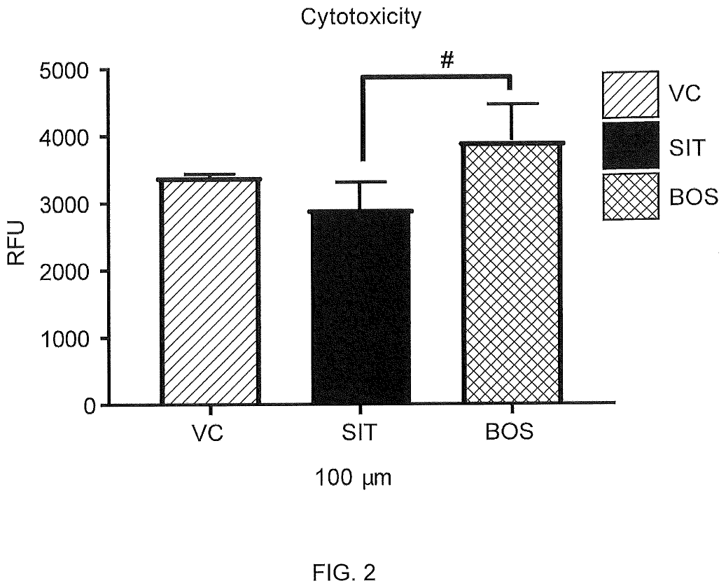

The data from Table 1B for the 100 μM concentrations of vehicle control, sitaxentan, and bosentan are presented as bar graphs with statistical analyses in FIG. 2.

These results show that neither bosentan nor sitaxentan was significantly more cytotoxic than the vehicle control; however, bosentan was significantly more cytotoxic than sitaxentan in TGF-β1 induced male normal human dermal fibroblasts.

For apoptosis:

The data are presented in Table 1C as the relative light units (RLUs) as a measure of cell apoptosis.

TABLE 1C
Apoptosis

| | Concentration of Test Material | | | | |
|---|---|---|---|---|---|
| | 1 μM | 3 μM | 10 μM | 30 μM | 100 μM |
| VC | 36833.3 | 40552.8 | 37338.7 | 37284.0 | 35834.2 |
| SIT | 37593.4 | 39162.8 | 39512.0 | 46053.5 | 51050.2 |
| BOS | 39366.8 | 38639.8 | 38586.7 | 42825.2 | 43330.7 |

VC = vehicle control,
SIT = sitaxentan,
BOS = bosentan

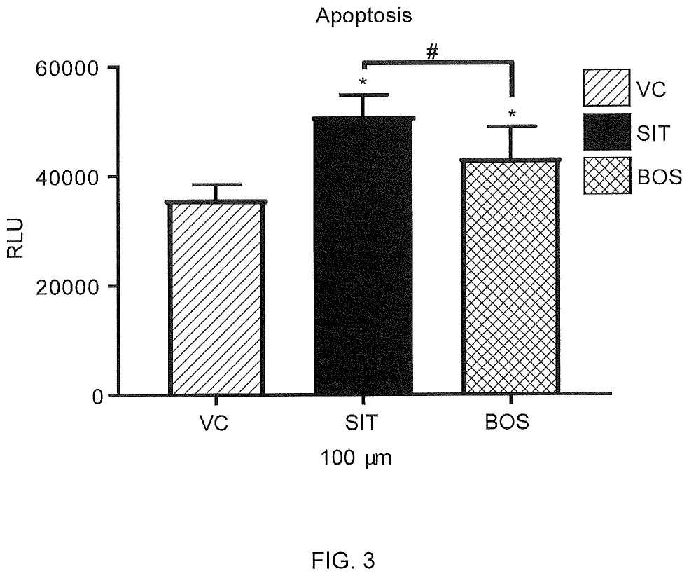

The data from Table 1C for the 100 μM concentrations of vehicle control, sitaxentan, and bosentan are presented as bar graphs with statistical analyses in FIG. 3.

These results show that apoptosis of TGF-β1 induced male normal human dermal fibroblasts was elevated after treatment with both sitaxentan and bosentan, but with sitaxentan having a significantly more potent effect than bosentan.

### Example 2

### Effect of Sitaxentan on Human Melanocytes

The effect of sitaxentan on human melanocytes was measured. For these assays cells were grown for 24 hours in the presence of vehicle control, sitaxentan, and bosentan. The appropriate assay reagents and measuring techniques were used as indicated herein.

Melanocytes, from either Caucasian or African American donors, were grown to confluence in 6-well tissue culture plates using LLCT DermaLife Ma complete medium and treated with compounds for 24 hours.

Treated cells were removed from the plate with 0.25% trypsin for 10 minutes and then centrifuged at 0.4 g for 5 minutes.

The resulting cell pellet was resuspended in 50 μL of 1M NaOH/10% DMSO solution.

The resultant suspension was vortexed for 30 seconds.

The suspension was then baked in a drying oven at 80° C. for 1 hour.

The suspension was vortexed again for 30 seconds and then transferred to a black half-area 96-well plate and read in a UV/VIS spectrophotometer at 400 nm.

Data were analyzed using GraphPad Prism 7.

For melanin content, data are presented in Table 2 as the absorbance at 400 nm as a measure of melanin content.

TABLE 2
Melanin Content

| | |
|---|---|
| 30 μM | 100 μM |

Caucasian Melanocytes

| | | |
|---|---|---|
| VC | 0.847 | 0.844 |
| SIT | 0.579 | 0.544 |
| BOS | 0.633 | 0.703 |

African American Melanocytes

| | | |
|---|---|---|
| VC | 0.490 | 0.586 |
| SIT | 0.471 | 0.46 |
| BOS | 0.511 | 0.497 |

VC = vehicle control,
SIT = sitaxentan,
BOS = bosentan

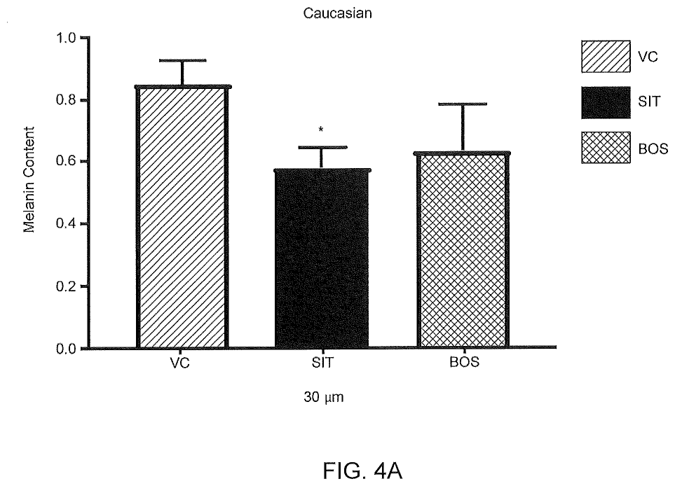

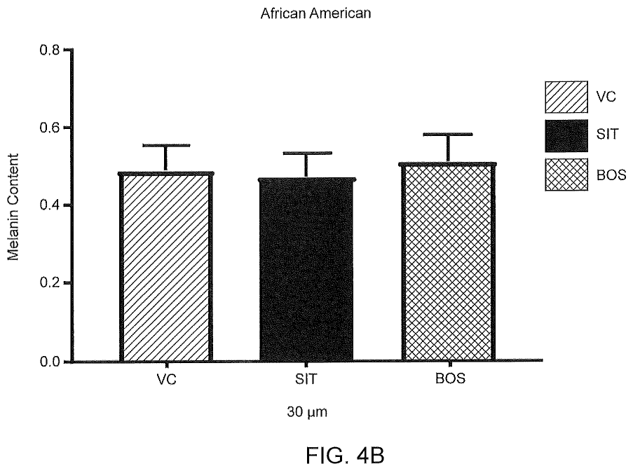

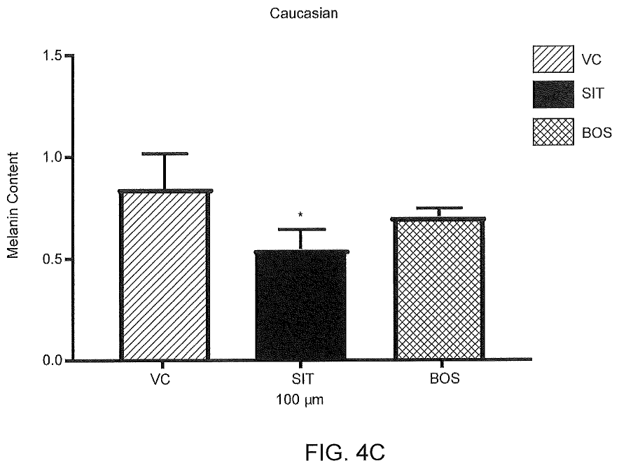

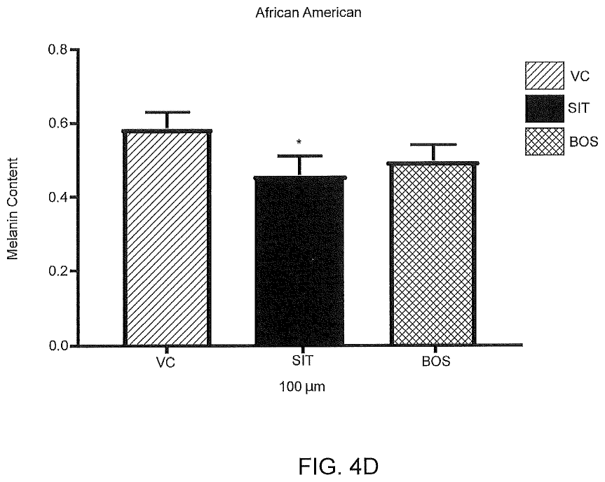

The data from Table 2 for the 30 and 100 μM concentrations of vehicle control, sitaxentan, and bosentan are presented as bar graphs with statistical analyses in FIGS. 4A, 4B, 4C, and 4D.

These results show that bosentan had no effect on melanin content in either African Americans or Caucasians, while sitaxentan significantly decreased melanin content in Caucasians and in African Americans.

### Example 3

### Preparation of a Composition for Topical Delivery

Sitaxentan sodium is mixed with ethanol to provide a 1% solution or suspension based on the weight of the sitaxentan active.

This composition is useful for topical administration to a human patient or animal for the treatment of conditions such as pigmentation disorders or irregularities.

### Example 4

### Preparation of a Suncsreen Composition for Topical Delivery

Sitaxentan sodium is mixed with a 50/50 mixture of ethanol and isopropanol to provide a 1% solution or suspension based on the weight of the sitaxentan active. The sunscreen material avobenzone is added and blended in at the desired level, such as at 3% to 10% by weight of the final composition, to provide a sunscreen composition containing sitaxentan sodium.

This composition is useful for topical administration to a human patient or animal for the treatment of conditions such as pigmentation disorders or irregularities and also to protect the skin from the effects of further exposure to sunlight or other sources of UV radiation.

### Incorporation by Reference

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

Equivalents

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight.

1. A method for treating hyperpigmentation or a pigmentation disorder or irregularity comprising locally or topically applying a therapeutically effective amount of a selective endothelin-A (ET-A) receptor antagonist or inhibitor to a mammal in need thereof.

2. A method according to claim 1 wherein the selective endothelin-A (ET-A) receptor antagonist or inhibitor has a selectivity of at least two-fold over endothelin-B (ET-B), or a selectivity of at least five-fold over endothelin-B (ET-B)), or a selectivity of at least ten-fold over endothelin-B (ET-B)), or a selectivity of at least 100-fold over endothelin-B (ET-B)), or a selectivity of at least 1000-fold over endothelin-B (ET-B)), or a selectivity of at least 5000-fold over endothelin-B (ET-B).

3. (canceled)

4. (canceled)

5. (canceled)

6. (canceled)

7. (canceled)

8. A method according to claim 1 wherein the selective endothelin-A (ET-A) receptor antagonist or inhibitor is sitaxentan or a pharmaceutically acceptable salt thereof.

9. A method according to claim 1 wherein the mammal is a human patient.

10. A method according to claim 1 wherein the pigmentation disorder or irregularity is selected from postinflammatory hyperpigmentation, erythema dyschromicum perstans, lichen planus pigmentosus, melasma, lentigo, age spots, freckling, vitiligo, albinism, acanthosis nigricans, incontinentia pigmenti, progressive pigmentary purpura, xeroderma pigmentosum, café au lai spots or macules, cholasma, liver spots, Addison Disease, melanocytic naevi, sebhorreic keratosis, melanoma, basal cell carcinoma, pityriasis alba, pityriasis versicolor, idiopathic guttate hypomelanosis, progressive macular hypomelanosis, urticarial pigmentosa, and pigmentation changes caused by drug reactions, infections, burns, and chemical damage, and combinations thereof.

11. A method according to claim 8 wherein the pharmaceutically acceptable salt is selected from an alkali metal salt, an alkaline earth metal salt, and an ammonium salt.

12. A method according to claim 11 wherein the alkali metal salt is selected from lithium, sodium, and potassium.

13. A method according to claim 11 wherein the alkali metal salt is sodium.

14. (canceled)

15. A method according to claim 1 wherein the selective endothelin-A (ET-A) receptor antagonist or inhibitor is applied at least one daily.

16. A method according to claim 1 wherein the selective endothelin-A (ET-A) receptor antagonist or inhibitor is applied at least twice daily.

17. A method according to claim 1 wherein the selective endothelin-A (ET-A) receptor antagonist or inhibitor is applied at least once weekly.

18. A method according to claim 1 wherein the selective endothelin-A (ET-A) receptor antagonist or inhibitor is applied at least twice weekly.

19. A method according to claim 1 wherein the selective endothelin-A (ET-A) receptor antagonist or inhibitor is applied at least once daily until the pigmentation disorder or irregularity is treated.

20. A method according to claim 1 wherein the selective endothelin-A (ET-A) receptor antagonist or inhibitor is applied from a pharmaceutically acceptable composition.

21. A method according to claim 1 for treating a pigmentation disorder or irregularity, comprising locally or topically applying a pharmaceutically acceptable composition comprising a therapeutically effective amount of sitaxentan or a pharmaceutically acceptable slat thereof.

22. (canceled)

23. (canceled)

24. (canceled)

25. (canceled)

26. (canceled)

27. A method according to claim 21 wherein the composition further comprises one or more sunscreen actives.

28. A method according to claim 27 wherein the sunscreen active is selected from aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, Zinc oxide, and combinations thereof.

29. (canceled)

30. (canceled)

31. A method according to claim 21 in the form of a unit dosage composition.

32. A method according to claim 31 wherein the unit dosage comprises from about 0.01 to about 1000 mg of sitaxentan or a pharmaceutically acceptable salt thereof, based on the weight of the sitaxentan active.

33. A method according to claim 31 wherein the unit dosage comprises from about 0.001% to about 25% by weight sitaxentan or a pharmaceutically salt thereof, based on the weight of the sitaxentan active, or from about 0.01% to about 10% by weight sitaxentan or a pharmaceutically acceptable salt thereof, based on the weight of the sitaxentan active, or from about 0.1% to about 5% by weight sitaxentan or a pharmaceutically acceptable salt thereof, based on the weight of the sitaxentan active, or from about 0.2% to about 3% by weight sitaxentan or a pharmaceutically acceptable salt thereof, based on the weight of the sitaxentan active.

34. (canceled)

35. (canceled)

36. (canceled)

37. A method according to claim 31 wherein the unit dosage demonstrates at least one of the following pharmacokinetic parameters selected from a Cₘₐₓ less than about 13 μg/ml, or a Cₘₐₓ less than about 7 μg/ml, or an AUC less than about 40 μg hr/ml.

38. (canceled)

39. A composition for treating hyperpigmentation or a pigmentation disorder or irregularity for local or topical delivery comprising a therapeutically effective amount of a selective endothelin-A (ET-A) receptor antagonist or inhibitor and a pharmaceutically acceptable carrier.

40. A composition according to claim 39 wherein the selective endothelin-A antagonist or inhibitor is sitaxentan or a pharmaceutically acceptable salt thereof
